(19) United States
(12) Patent Application Publication
(10) Pub. No.: US 2017/0087318 A1
(43) Pub. Date: Mar. 30, 2017

(54) CUFFED TUBES

(71) Applicant: SMITHS MEDICAL INTERNATIONAL LIMITED, Kent (GB)

(72) Inventors: Timothy Bateman, Kent (GB); Andrew Thomas Jeffrey, Kent (GB); Christopher Stratton Turnbull, Kent (GB)

(73) Assignee: SMITHS MEDICAL INTERNATIONAL LIMITED, Kent (GB)

(21) Appl. No.: 15/124,105

(22) PCT Filed: Feb. 6, 2015
(86) PCT No.: PCT/GB2015/000047

§ 371 Date: Sep. 7, 2016

(30) Foreign Application Priority Data
Mar. 8, 2014 (GB) 1404220.4
Aug. 6, 2014 (GB) 1413918.2

(51) Int. Cl. A61M 16/04

(52) U.S. Cl. CPC A61M 16/0434; A61M 16/0465; A61M 2207/00; B29C 69/00

(57) ABSTRACT

A cuffed tracheostomy tube has a sealing cuff 10, 10′, 100, 100′ attached to a shaft 1, 1, 1″ of the tube towards its patient end by a rear end collar 30, 55, 103 bonded with the outside of the shaft. In one embodiment the patient end collar 31, 31′ is folded unattached around the patient end face 60, 60′ of the shaft and is attached to the inside of the tube. When inflated, the cuff inflates slightly away from the patient end face to cushion the tip of the tube. In another embodiment the cuff 100 is formed integrally with the shaft 51. In a further embodiment the cuff 100′ has an integral portion 102 of a soft material that forms a patient end extension of the shaft 1″.

This invention relates to medico-surgical tube of the kind having a shaft and an inflatable sealing cuff on its outside towards its forward, patient end that is adapted, during use, to provide a seal with a body cavity, the cuff including a collar at its rear, machine end attached with the outside of the shaft.

Tracheal tubes are used to supply ventilation and anaesthetic gases to a patient, such as during surgery. The tracheal tube may be inserted via the mouth or nose, in the case of an endotracheal tube, or may be inserted via a surgically-made tracheostomy opening in the neck, in the case of a tracheostomy tube. Most, but not all, tracheal tubes have some form of a seal on their outside which forms a seal between the outside of the tube and the inside of the trachea so that gas flow is confined to the bore of the tube and cannot flow around the outside of the tube, between the tube and the trachea. The most common form of seal is provided by an inflatable cuff that is inflated and deflated via a small bore lumen extending rearwardly along the tube and connected towards its rear end to an inflation line terminated by an inflation indicator, valve and connector. These inflatable cuffs may be of the high-volume/low-pressure kind where the cuff is formed of a flexible material moulded with a natural annular or doughnut shape that is inflated without stretching by relatively low-pressure gas supplied via the inflation line. Alternatively, the cuff may be of the low-volume/high-pressure kind where the cuff is of an elastic material that lies close to the tube shaft when uninflated but is inflated and stretched to a larger diameter by relatively high pressure gas supplied via the inflation line. Cuffs of differing shapes and configurations are described, for example, in U.S. Pat. No. 7,987,851, GB2356571, U.S. Pat. No. 8,307,830 and U.S. Pat. No. 8,434,488. Cuffs are also used on other tubes where it is necessary to form a seal with a body cavity.

One problem with tracheal tubes, especially tracheostomy tubes, is that the tip of the tube may contact the tracheal wall during use and cause damage to the tracheal tissue.

It is an object of the present invention to provide an alternative cuffed tube.

According to one aspect of the present invention there is provided a medico-surgical tube of the above-specified kind, characterised in that the cuff towards its patient end extends over the patient end of the shaft.

The patient end of the cuff may extend unattached around the patient end face of the shaft such that, when the cuff is inflated, it inflates across the patient end face to cushion the patient end face. The patient end of the cuff may be attached with the inside of the shaft by a patient end collar. The tube may include an inner cannula extending within the shaft, the patient end collar extending along a first region towards the patient end of the inside of the shaft where it is unattached with the shaft and a second region to the rear of the first region where it is attached with the shaft such that the cuff along the first region is inflatable slightly away from the inside surface of the shaft into contact with the outside of the inner cannula.

Alternatively, the patient end of the cuff may provide a tubular portion that provides an extension of the shaft of the tube. The tubular portion may have a wall thickness greater than that of the inflatable sealing portion of the cuff, the internal surface of the tubular portion forming a smooth continuation of the internal surface of the shaft. The forward end of the shaft and the rear end of the tubular portion may have cooperating locating formations. The cooperating locating formations may comprise a recess on one part and a projection on the other part.

According to another aspect of the present invention there is provided a medico-surgical tube including a tubular shaft and an inflatable cuff towards the patient end of the shaft, characterised in that the tube has a soft tip formed integrally with the cuff as a single piece.

According to a further aspect of the present invention there is provided a medico-surgical tube including a tubular shaft and an inflatable cuff towards the patient end of the shaft, the cuff being formed integrally as a single piece with the shaft.

One end of the cuff may be integrally attached with the shaft and the other end of the cuff may be bonded to the outside of the shaft.

According to a fourth aspect of the present invention there is provided a method of forming a cuffed tube including the steps of moulding a shaft having an integral sheath moulded therewith, the sheath being integrally attached at one end with the patient end of the shaft and having a free end at its opposite end, the sheath having a thinner wall than that of the shaft, folding the free end of the sheath rearwardly along the outside of the patient end of the shaft and attaching the free end of the sheath to the outer wall of the shaft to form an inflatable cuff around the patient end of the tube.

According to a fifth aspect of the present invention there is provided a cuffed tube made by a method according to the above fourth aspect of the present invention.

A tracheostomy tube according to the present invention will now be described, by way of example, with reference to the accompanying drawings, in which:

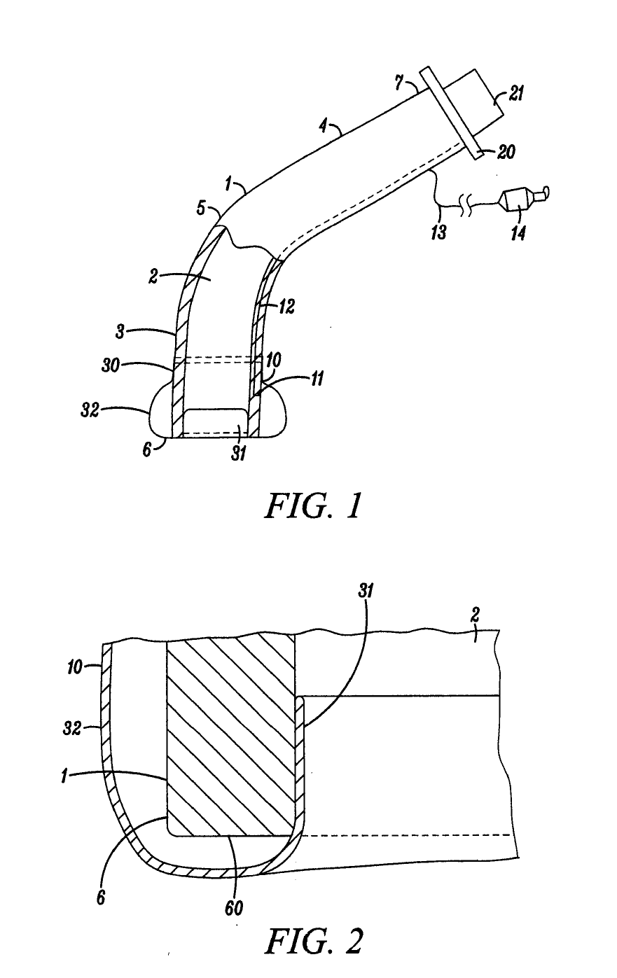

FIG. 1 is a partly cross-sectional side elevation view of the tube;

FIG. 2 is an enlarged cross-sectional view through a part of the patient end of the tube shown in FIG. 1;

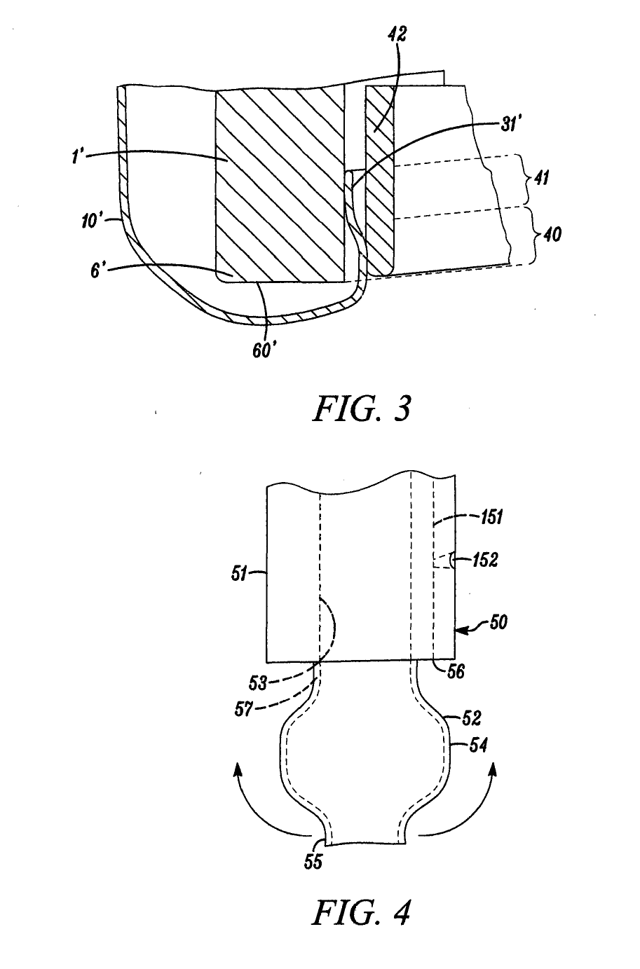

FIG. 3 is an enlarged cross-sectional view through a part of the patient end of an alternative tube including an inner cannula;

FIG. 4 is a side elevation view of the patient end a moulding at a preliminary stage in manufacture of a cuffed tube;

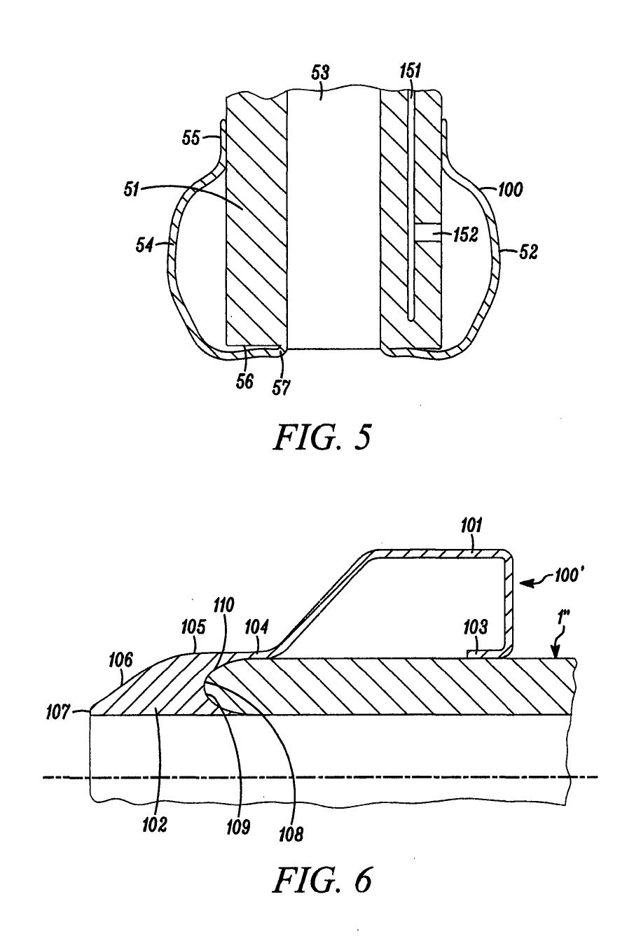

FIG. 5 is a cross-sectional side elevation view of the patient end of the moulding shown in FIG. 4 at a subsequent stage of manufacture; and

FIG. 6 is a cross-sectional side elevation view of an alternative tube.

With reference to FIGS. 1 and 2, the tracheostomy tube includes a tubular shaft 1 having a bore 2 extending along its length. The tube is formed with a relatively straight patient end portion 3 and a relatively straight machine end portion 4 linked by a curved intermediate portion 5 so that the patient and machine ends 6 and 7 are angled at about 100° to one another. The shaft 1 is extruded or moulded from a plastics or rubber material such as PVC, polyurethane or silicone. Towards its patient end 6 the tube has sealing means provided by an inflatable cuff 10 embracing the shaft 1. The cuff 10 is of the high-volume/low-pressure kind so that it has a relatively floppy shape when deflated but, when inflated, it fills out at low pressure to a diameter just larger than the internal diameter of the trachea, so that it contacts the inside of the trachea with low pressure. Alternatively, a low volume, elastic cuff that lies close to the tube shaft when deflated could be used. The cuff 10 is moulded to the desired inflated shape from a plastics or rubber material such as PVC, polyurethane or rubber. The cuff 10 is attached to the shaft 1 at opposite ends over an opening 11 on the outer surface of the shaft into an inflation lumen 12 extending along the shaft within its wall thickness. The inflation lumen 12 is connected towards the rear end 7 of the tube with a small-bore inflation line 13 that is terminated by a combined inflation indicator, valve and connector 14.

At its rear, machine end 7 the tube has a flange 20, to which a neck strap (not shown) is attached, and a standard 15 mm female coupling 21.

The cuff 10 is attached to the outside of the shaft 1 by a machine end collar 30 and a patient end collar 31. The machine end collar 30 is attached to the shaft 1 in the usual way, by extending the collar externally beyond the inflatable portion 32 of the cuff 10 and bonding its inner surface to the outside of the shaft, such as with an adhesive or solvent or by a heat bonding method.

The patient end collar 31, however, is attached in a different manner in that it is folded around the patient end 6 of the shaft 1 and turned inside the bore 2 of the shaft so that what was originally the inwardly-facing surface of the collar now faces outwardly. The now outwardly-facing surface of the collar 31 contacts and is bonded with the surface of the bore 2 towards the patient end 6, such as by means of a solvent, adhesive or by a heat bonding process. The patient end collar 31 is, therefore, only attached with the inside of the shaft 1. A cylindrical mandrel (not shown) may be inserted in the patient end of the shaft 1 during the bonding process to hold the collar 31 into intimate contact with the inside surface of the shaft.

In this way, the cuff 10 extends around the end face 60 of the shaft 1 at its patient end 6 to cover it and thereby prevent it from directly contacting patient tissue. When inflated, the cuff 10 expands close to the patient end tip 6 of the shaft 1, thereby providing a cushion or buffer to soften it and reduce tissue trauma. Preferably, the cuff 10 is not bonded to the end face 60 itself so that the cuff material can expand slightly away from the shaft 1 over the end face and thereby increase the cushioning effect. The cuff 10 could, however, be bonded with the end face 60 and still provide some cushioning effect because the cuff would bulge down slightly beyond the patient end of the shaft when inflated. The end face 60 of the shaft 1 is shown as square, that is, extending at right angles to the axis. It could, however, be rounded or angled to form a tapered tip surface, which would be advantageous when used in percutaneous insertion techniques.

There are two possible alternative arrangements for bonding the patient end collar 31 to the inside surface of the bore 2. In one arrangement, shown in FIG. 2, the patient end collar 31 is bonded to the surface of the bore 2 along the entire area where it overlaps the inside of the shaft 1. In the other arrangement, shown in FIG. 3, the collar 31′ is only bonded with the shaft 1′ along a part of the area of overlap and, in particular, it is not bonded with the shaft along a first annular region 40 towards patient end 6′ of the shaft but is only bonded over a second region 41 to the rear of the first region. In this way, when the cuff 10′ is inflated, in addition to it inflating over the end face 60′, it also inflates slightly inside the shaft 1′ over the first region 40. This can be an advantage where the tube is used with an inner cannula 42 because the inflated internal region of the cuff 10′ can be arranged to inflate into sealing contact with the outside of the inner cannula and thereby seal any gas passage between the outside of the inner cannula and the inside of the outer shaft 1′.

It would be possible for the rear, machine end collar of the cuff to be inverted inside the inflated portion, in the manner described in U.S. Pat. No. 5,201,310, especially if the tube had a suction line for aspirating secretions that collect above the cuff.

In the arrangements described above, the cuffs are made separately from the shaft and are subsequently attached to the shaft. It is possible, however, to form the cuff 100 integrally with the tube shaft in the manner shown in FIGS. 4 and 5. With reference first to FIG. 4 there is shown a moulded component 50 including a tubular, shaft region 51 having a relatively thick wall (from which the main shaft of the tube will be formed). An inflation lumen 151 extends within the thickness of the wall and along the length of the tubular region. An opening 152 is subsequently cut into the lumen 151 from the outer surface close to its lower, patient end. The moulded component 50 also includes a thin-walled tubular sheath 52 (from which the cuff will be formed) extending integrally as one piece from the lower, patient end of the tubular region 51 so that it is integrally attached with the shaft region 51 at one end 57. The bore 53 of the tubular region 51 continues in a stepless fashion through the sheath 52. Because the wall thickness of the sheath 52 is much less than that of the tubular region 51, the external diameter of the sheath is less than that of the tubular region thereby forming an annular step 56 at the end of the tubular region around the outside of the sheath, corresponding to the patient end face of the shaft. The sheath 52 is integrally attached with the shaft region 51 around the inner edge of the step 56. The sheath 52 may have an enlarged diameter region 54 along its length so that its external diameter is a maximum about midway along its length. The lower end of the sheath has a cylindrical, collar region 55 of constant diameter approximately equal to the external diameter of the tubular region 51.

The cuff 100 is formed by stretching the lower end 55 of the sheath 52 and turning it inside out around the outside of the lower end of the tubular, shaft region 51, as shown in FIG. 5. What was the outer surface of the sheath 52 becomes its inside surface, the collar region 55 being located rearmost and embracing the outer surface of the shaft region 51 at a location to the rear of the opening 152 into the inflation lumen 151. The collar region 55 is bonded with the outer surface of the shaft region 51 by any conventional means, such as an adhesive, solvent or by a weld or other thermal bonding process. When inflated, the cuff 100 expands mainly in the enlarged region 54 but also right up to the patient end of the shaft 51 and also inflates slightly across the step surface 56 so that the cuff provides an effective buffer at the patient end of the tube to reduce the risk of trauma.

This manufacturing technique may be easier than attaching one end of a preformed cuff to the inside of the shaft. It also avoids any risk of adhesive or bonding agent seeping across the end face of the shaft, ensuring that the cuff can be inflated away from the end face. The plastics material used in this manufacturing technique needs to be selected such that it provides suitable characteristics both for the tube shaft and for the cuff. The cuff may be of an elastic type that lies close to the tube shaft when deflated and stretches away from the shaft when inflated.

An inflatable cuff on a tube could be used to provide cushioning of the tip of the tube in ways other than by inflating over the end of the shaft.

FIG. 6, for example, shows a cuff member 100 that is an integral moulding of a soft plastics material that is softer than the material forming the shaft 1″. The cuff member 100 provides both an inflatable sealing portion 101 and a soft tip portion 102 forming a soft, conformable tip or extension to the shaft 1 of the tube. The sealing portion 101 may be of any conventional shape, such as rounded, cylindrical, tapered, corrugated or the like and is formed of a relatively thin wall so that the sealing portion can be collapsed and expanded away from the wall of the shaft 1″ by gas pressure supplied along an inflation lumen (not shown) extending within the wall of the shaft.

At its rear, machine end the wall of the sealing portion 101 is folded inside the inflatable portion to form a collar 103 that is attached to the outer surface of the shaft 1″. Alternatively, the collar could extend externally and be attached with the shaft outside the inflatable portion. At the opposite end of the sealing portion 101 its wall extends forwardly along the outside of the shaft 1 to form an optional second, patient end attachment collar 104.

The patient end collar 104 extends to the end of the shaft 1″ where it continues as the tip portion 102. The tip portion 102 has a rear, machine end cylindrical portion 105 with a wall thickness that is equal to the wall thickness of the shaft 1″ plus the wall thickness of the collar 104. The cylindrical portion 105 extends axially along about half the length of the tip portion 102. The forward half of the tip portion 102 tapers on its external surface 106 so that its wall thickness reduces forwardly along its length, with the forward end 107 of the tip portion 102 being rounded. The lateral, rear end face 108 of the tip portion 102 is moulded with a locating formation in the form of a concave, annular recess 109. The forward, patient end face 110 of the shaft 1″ is shaped to provide a cooperating locating formation in the form of a convex rounded annular projection of the same dimensions as the recess 109 on the tip portion 102. The two locating formations 109 and 110 cooperate with one another during assembly to help centralise the tip portion 102 on the patient end of the shaft 1″.

By forming the cuff member 100′ of a softer material than the shaft 1″, the tip of the tube is softer than would be the case without the added tip portion. The tapering forward end 106 of the tip portion 102 also makes its patient end more flexible. This helps reduce trauma to tissue during insertion and use of the tube. Forming the inflatable sealing cuff integrally with the soft tip has advantages in that both the soft tip and the sealing cuff can be assembled in a single operation, instead of having to make two separate operations as would be the case if a separate soft tip were used. There is a further advantage in that there is a greater security from the risk that the tip could become detached because, in effect, the tip is additionally anchored to the external surface of the shaft by the cuff instead of just being attached to the end tip of the shaft.

The invention could be used in any cuffed tube, not just tracheal tubes, and is not limited to tubes for human use but could be used on cuffed veterinary tubes.

1-13. (canceled)

14. A medico-surgical tube having a shaft and an inflatable sealing cuff on its outside towards its forward, patient end that is adapted, during use, to provide a seal with a body cavity, the cuff including a collar at its rear, machine end attached with the outside of the shaft, characterized in that the cuff towards its patient end extends over the patient end of the shaft.

15. A tube according to claim 14, characterized in that the patient end of the cuff extends unattached around the patient end face of the shaft such that, when the cuff is inflated, it inflates across the patient end face to cushion the patient end face.

16. A tube according to claim 14, characterized in that patient end of the cuff is attached with the inside of the shaft by a patient end collar.

17. A tube according to claim 16, characterized in that the tube includes an inner cannula extending within the shaft and that the patient end collar extends along a first region towards the patient end of the inside of the shaft where it is unattached with the shaft and a second region to the rear of the first region where it is attached with the shaft such that the cuff along the first region is inflatable slightly away from the inside surface of the shaft into contact with the outside of the inner cannula.

18. A tube according to claim 14, characterized in that the patient end of the cuff provides a tubular portion that provides an extension of the shaft of the tube.

19. A tube according to claim 18, characterized in that the tubular portion has a wall thickness greater than that of the inflatable sealing portion of the cuff, and that the internal surface of the tubular portion forms a smooth continuation of the internal surface of the shaft.

20. A tube according to claim 18, characterized in that the forward end of the shaft and the rear end of the tubular portion have cooperating locating formations.

21. A tube according to claim 20, characterized in that the cooperating locating formations comprise a recess on one part and a projection on the other part.

22. A medico-surgical tube including a tubular shaft and an inflatable cuff towards the patient end of the shaft, characterized in that the tube has a soft tip formed integrally with the cuff as a single piece.

23. A medico-surgical tube including a tubular shaft and an inflatable cuff towards the patient end of the shaft, characterized in that the cuff is formed integrally as a single piece with the shaft.

24. A tube according to claim 23, characterized in that one end of the cuff is integrally attached with the shaft and the other end of the cuff is bonded to the outside of the shaft.

25. A method of forming a cuffed tube including the steps of moulding a shaft having an integral sheath moulded therewith, the sheath being integrally attached at one end with the patient end of the shaft and having a free end at its opposite end, the sheath having a thinner wall than that of the shaft, folding the free end of the sheath rearwardly along the outside of the patient end of the shaft and attaching the free end of the sheath to the outer wall of the shaft to form an inflatable cuff around the patient end of the tube.

26. A cuffed tube made by moulding a shaft having an integral sheath moulded therewith, the sheath being integrally attached at one end with the patient end of the shaft and having a free end at its opposite end, the sheath having a thinner wall than that of the shaft, folding the free end of the sheath rearwardly along the outside of the patient end of the shaft and attaching the free end of the sheath to the outer wall of the shaft to form an inflatable cuff around the patient end of the tube.
